(19) United States
(12) Patent Application Publication
(10) Pub. No.: US 2026/0283586 A1
(43) Pub. Date: Sep. 24, 2026

(54) System and Method for Remote Stethoscope for Telemedicine

(71) Applicants: Dissanayake Gunasekera, Houston, TX (US); Yasantha Rajakarunanayake, San Ramon, CA (US)

(72) Inventors: Dissanayake Gunasekera, Houston, TX (US); Yasantha Rajakarunanayake, San Ramon, CA (US)

(21) Appl. No.: 19/083,459

(22) Filed: Mar. 19, 2025

(51) Int. Cl. A61B 7/04; A61B 5/00; G16H 10/60; G16H 40/67

(52) U.S. Cl. CPC A61B 7/04; A61B 5/0022; A61B 5/7264; G16H 10/60; G16H 40/67

(57) ABSTRACT

The invention, titled “A System and Method for Remote Stethoscope for Telemedicine,” introduces a remotely operable stethoscope system to enhance telemedicine by enabling real-time audio transmission of medical sounds. The system includes a low-frequency audio pick-up device on the patient side and a high-fidelity sound playback device on the doctor's side, enabling remote consultations with improved diagnostic capabilities. Advanced features include Angle of Arrival (AoA) analysis, stereo 3D sound, AI-assisted diagnostics, and secure recording and playback capabilities.

### CLAIM OF PRIORITY

This application claims priority to U.S. Provisional Patent Application No. 63/567,952, filed on Mar. 21, 2024, titled “A System and Method for Remote Stethoscope for Telemedicine.” This provisional application is incorporated herein by reference in its entirety.

### FIELD OF THE DISCLOSURE

The present invention pertains to telemedicine, specifically focusing on a remote stethoscope system that enables real-time audio transmission of medical sounds for improved remote patient diagnosis.

### BACKGROUND

The widespread adoption of telemedicine has been accelerated by global health challenges. However, a significant limitation remains: the inability to capture and transmit medical sounds like heart rhythms and lung sounds in real time. Without this capability, remote consultations often lack the diagnostic accuracy required for effective patient care.

Traditional telemedicine solutions rely on patient-reported symptoms and visual assessments via video conferencing, which may not always provide sufficient information for accurate diagnoses. A real-time, high-fidelity audio transmission system can significantly enhance remote medical assessments, reducing the need for follow-up in-person visits and improving overall healthcare efficiency.

### SUMMARY OF THE INVENTION

The invention introduces rSteth, a remote stethoscope system that facilitates real-time medical sound transmission for telemedicine. The system includes the following core components:
A low-frequency audio pick-up device (patient-side) for capturing vital medical sounds.
A transmission gateway using Bluetooth, Wi-Fi, or wired connections.
A high-fidelity playback device (doctor-side) for real-time audio analysis.
AI-powered diagnostic tools for preliminary assessments.
Secure cloud storage and Electronic Health Record (EHR) integration.
Angle of Arrival (AoA) analysis for enhanced audio spatial representation.
Smartphone and smartwatch compatibility for expanded usability.
HIPAA-compliant security measures for data transmission and storage.

### BRIEF DESCRIPTION OF THE DRAWINGS

The invention is further illustrated in the accompanying drawings:

FIG. 1: Overview of the rSteth system architecture.

FIG. 2: System block diagram showing patient-side and doctor-side components.

FIG. 3: Functional flowchart detailing data processing and transmission.

FIG. 4: Diagram illustrating the AI-assisted diagnostic workflow.

FIG. 5: Diagram illustrating the secure data storage and transmission system.

### DETAILED DESCRIPTION

### System Architecture

The rSteth system is designed to facilitate high-quality remote auscultation, enabling real-time transmission and analysis of patient medical sounds. The system is composed of:

### Patient-Side Components

Digital stethoscope with a high-sensitivity microphone for capturing low-frequency medical sounds.
Audio transmission module supporting Bluetooth, Wi-Fi, and wired connections.
Power-efficient design with rechargeable battery or USB-powered operation.
Antimicrobial surface coatings to prevent microbial contamination.

### Doctor-side Components:

High-fidelity playback system optimized for clear, real-time auscultation.
Angle of Arrival (AoA) feature for stereo 3D sound representation.
Integration with standard medical audio analysis software.
Wireless connectivity for mobile use on tablets and smartphones.

### Software Features

AI-assisted diagnostics to detect abnormalities in heart and lung sounds.
Secure cloud-based recording and playback with HIPAA compliance.
Integration with Electronic Health Records (EHR) for seamless data access.

### User Interfaces

Dedicated smartphone and smartwatch applications for both patients and doctors.
Graphical sound visualization tools for enhanced diagnostic assessment.
Configurable alerts and notifications for real-time patient monitoring.

### Functional Workflow

The rSteth system operates using the following workflow:
1. Patient activates the remote stethoscope, which captures heart and lung sounds.
2. Audio signals are transmitted securely via Bluetooth, Wi-Fi, or wired interfaces.
3. Doctor receives the transmitted audio, which is played back with high fidelity.
4. Angle of Arrival (AoA) analysis reconstructs a 3D stereo sound field for precise auscultation.
5. AI-powered diagnostics process the audio and highlight potential medical concerns.
6. Securely stored data allows doctors to review, annotate, and integrate findings into EHRs.

### Example Systems and Methods

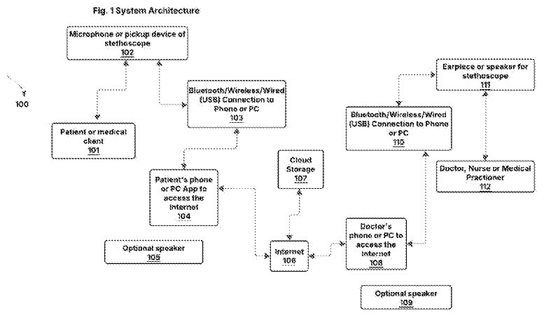

FIG. 1 illustrates an example process 100 for the rSteth system architecture that facilitates real-time medical sound transmission, according to some embodiments. The process begins with the patient or medical client (Step 101), who uses the stethoscope's microphone or pickup device (Step 102) to capture auscultatory sounds from the body. These sounds are then transmitted via a Bluetooth, wireless, or wired (USB) connection (Step 103) to the patient's phone or PC (Step 104), which serves as the interface for accessing the internet and further processing the audio. An optional speaker (Step 105) may be used for local playback if needed.

The system relies on internet connectivity (Step 106) to facilitate real-time or stored data transfer. Once recorded, the auscultatory data can be uploaded to cloud storage (Step 107), ensuring secure access and retrieval for medical professionals. On the doctor's side, the doctor's phone or PC (Step 108) connects to the internet to retrieve and analyze the patient's recorded sounds. Similar to the patient's setup, an optional speaker (Step 109) may be used for playback, and the device can receive data via Bluetooth, wireless, or wired (USB) connections (Step 110).

For direct listening, the doctor or medical practitioner (Step 112) can use an earpiece or speaker designed for stethoscope audio (Step 111) to ensure precise auscultation. This entire system enables seamless remote monitoring and diagnosis, allowing healthcare providers to assess patients' conditions efficiently from different locations.

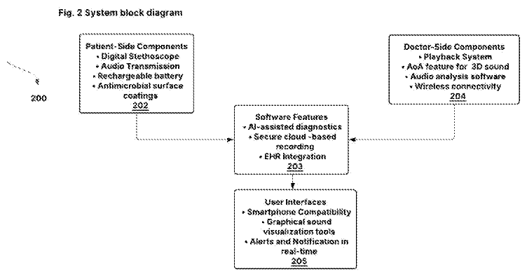

FIG. 2 illustrates an example system 200 for showing patient-side and doctor-side components, which outlines the key functional components that enable advanced auscultation and diagnostic capabilities. It begins with Patient-Side Components (Step 202), where a digital stethoscope captures auscultatory sounds and transmits them via an audio transmission system. The device is powered by a rechargeable battery and features antimicrobial surface coatings to ensure hygiene and minimize cross-contamination risks.

The next stage, Software Features (Step 203), enhances diagnostic accuracy through AI-assisted diagnostics, which analyze audio data for potential abnormalities. A secure cloud-based recording system allows for efficient data storage and retrieval, while EHR integration ensures seamless incorporation of recorded sounds into the patient's medical history.

On the Doctor-Side (Step 204), the system includes a playback system that enables physicians to review recorded sounds. An Angle of Arrival (AoA) feature for 3D sound enhances spatial audio perception, improving diagnostic precision. Additionally, audio analysis software aids in interpreting the sounds, and wireless connectivity ensures smooth data transmission between devices.

Finally, User Interfaces (Step 205) make the system accessible and user-friendly. Smartphone compatibility allows for mobile access to diagnostic data, while graphical sound visualization tools provide waveform and spectrogram representations for better analysis. The system also includes alerts and real-time notifications, ensuring that physicians are promptly informed of critical auscultatory findings. Together, these components create a comprehensive, AI-enhanced, and connected auscultation system designed to improve patient care.

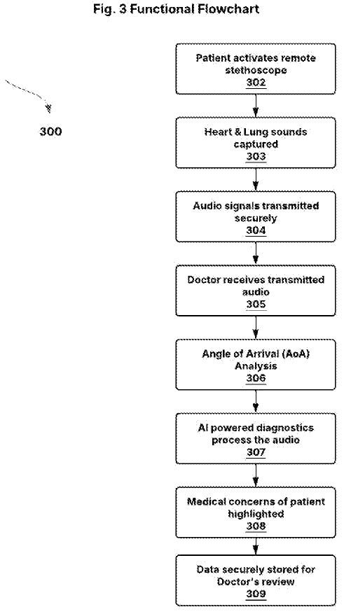

FIG. 3 illustrates an example data processing and transmission model, according to some embodiments. The data processing and transmission model 300 details the step-by-step process of data processing and transmission in the rSteth system, ensuring seamless remote auscultation and diagnostic analysis. The process begins when the patient activates the remote stethoscope (Step 302), initiating the recording session. The stethoscope then captures heart and lung sounds (Step 303), converting them into digital audio signals. These audio signals are transmitted securely (Step 304) via a wireless or wired connection, ensuring privacy and data integrity.

Once the transmission is complete, the doctor receives the transmitted audio (Step 305), allowing for remote evaluation. To enhance diagnostic precision, an Angle of Arrival (AoA) analysis (Step 306) is performed, enabling a 3D sound representation that improves spatial perception of auscultatory signals.

Next, the system applies AI-powered diagnostics to process the audio (Step 307), identifying potential anomalies in the recorded sounds. If any abnormalities are detected, the system highlights medical concerns (Step 308), flagging them for further evaluation by the healthcare provider.

Finally, all recorded and analyzed data is securely stored for the doctor's review (Step 309), ensuring easy access to patient records and facilitating follow-up assessments. This structured process enables accurate, remote auscultation with AI-assisted diagnostics, improving the efficiency of patient care.

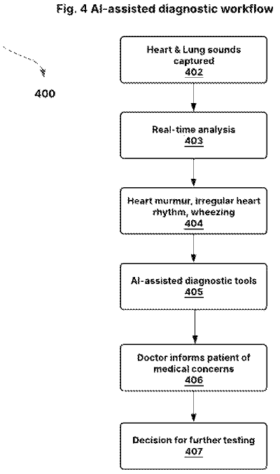

FIG. 4 illustrates an example workflow 400 for how the rSteth system leverages artificial intelligence to analyze heart and lung sounds and assist doctors in diagnosing potential medical conditions, according to some embodiments. The process begins with the capture of heart and lung sounds (Step 402) using the digital stethoscope.

These recorded sounds are then subjected to real-time analysis (Step 403), where the system immediately processes the audio data to detect any abnormalities.

During this analysis, the system identifies key cardiopulmonary issues such as heart murmurs, irregular heart rhythms, and wheezing (Step 404), which may indicate underlying medical conditions. The AI-assisted diagnostic tools (Step 405) further refine the analysis, providing insights that help the doctor interpret the findings accurately.

Based on the AI's analysis, the doctor informs the patient of any medical concerns (Step 406) detected in the recorded sounds. If necessary, the doctor then makes a decision for further testing (Step 407), such as ordering additional diagnostic procedures or referring the patient to a specialist for further evaluation.

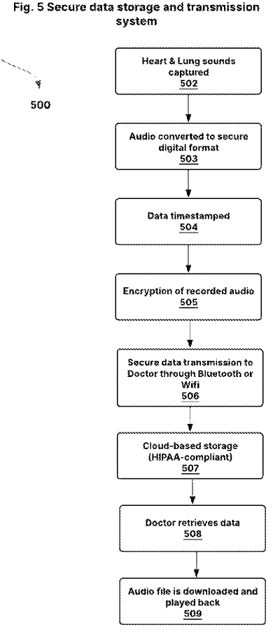

FIG. 5 illustrates an example process 500 by which the rSteth system ensures the confidentiality, integrity, and accessibility of patient auscultation data, according to some embodiments. The process begins with the capture of heart and lung sounds (Step 502) using the digital stethoscope. Once recorded, the audio is converted into a secure digital format (Step 503) to facilitate processing and transmission. Each recorded file is then timestamped (Step 504) to ensure proper documentation and tracking of patient data.

To protect sensitive medical information, the system encrypts the recorded audio (Step 505) before transmission, ensuring that only authorized healthcare providers can access it. The secure data transmission to the doctor (Step 506) occurs via Bluetooth or Wi-Fi, allowing for seamless real-time access or later retrieval.

For long-term storage and compliance with medical regulations, the encrypted data is stored in a HIPAA-compliant cloud-based system (Step 507), ensuring secure access and retrieval. The doctor can then retrieve the data (Step 508) at any time for review. Finally, the audio file is downloaded and played back (Step 509), enabling detailed analysis and comparison with previous recordings if necessary.

1. A system for real-time medical sound transmission, comprising:
A low-frequency audio pickup device for capturing patient medical sounds.
A data transmission module for wireless and wired transmission.
A high-fidelity playback system for remote auscultation.
Angle of Arrival (AoA) analysis for spatial sound representation.
AI-powered diagnostic tools for real-time analysis. Secure cloud storage and HIPAA-compliant encryption.

2. The system of claim 1, wherein multiple pickup devices are arranged for spatial sound analysis.

3. A method for embedding medical audio recording technology within a stethoscope chest piece.

4. The system of claim 1, integrating with EHR systems for seamless medical record access.

5. A system for detecting heart and lung sound abnormalities using AI-based analysis.

6. The system of claim 1, wherein smartphone and smartwatch applications provide enhanced usability.

7. A remote auscultation method that transmits medical sounds with secure cloud-based storage.
